(19) United States
(12) Patent Application Publication
(10) Pub. No.: US 2008/0161639 A1
(43) Pub. Date: Jul. 3, 2008

(54) Capsule medical apparatus and body-cavity observation method

(75) Inventors: Miho Katayama, Yokohama-shi (JP); Hironobu Takizawa, Tokyo (JP); Hironao Kawano, Tokyo (JP); Hidetake Segawa, Tokyo (JP); Hideo Ito, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Medical Systems Corporation, Tokyo (JP)

(21) Appl. No.: 11/646,964

(22) Filed: Dec. 28, 2006

(51) Int. Cl. A61B 1/00

(52) U.S. Cl. 600/104

(57) ABSTRACT

A posture and a position of a capsule endoscope 31 in a fluid 7 are made unstable and susceptible to an influence of a flow of the fluid 7 by constructing the capsule endoscope in such a way that the capsule endoscope 31 is equipped in a capsule casing having an approximately cylindrical trunk, a center of gravity of the capsule endoscope 31 is approximately in a volume center, and a specific gravity of the capsule endoscope 31 is approximately equal to that of the fluid 7 injected into a body cavity, thereby causing the capsule endoscope 31 to move to an intraluminal center using a difference of flow rate of the fluid 7 generated in large intestine and the like where a luminal diameter is large to stably capture desired intraluminal images by an imaging optical system.

### BACKGROUND OF THE INVENTION

1. Field of the Invention

The present invention relates to a capsule medical apparatus capable of, for example, capturing stable and desired wide-angle images along a flow of a fluid in the fluid injected into a subject and a body-cavity observation method.

2. Description of the Related Art

Recently, capsule endoscopes equipped with an imaging function and a radio communication function have emerged in a field of endoscopes. The capsule endoscopes are structured to move inside organs (body cavity) such as the esophagus, stomach, small intestine, and large intestine accompanying peristaltic movements thereof to successively capture images using the imaging function in an observation period after being swallowed through a mouth of a subject, a human body, for observation (examination) until being naturally discharged from a living body of the subject.

Here, a technique suitable for observation of the large intestine is disclosed in Patent Document 1 (WO 02/95351 (Japanese Unexamined Patent Application Publication (Translation of PCT Application) 2004-529718)), whereby a capsule endoscope is advanced fast to the large intestine in the body cavity by drifting the capsule endoscope in a fluid after the capsule endoscope being swallowed together with the fluid, with a specific gravity of the capsule endoscope set to about 1, which is equal to that of a surrounding liquid or water. Only areas near a body cavity wall surface can be observed if the capsule endoscope sticks to the body cavity wall surface, but according to Patent Document 1, an observation visual field can be secured for exhaustive observation because observations are made by drifting the capsule endoscope in the fluid.

However, there has been a problem that, when a conventional capsule endoscope moves inside a wide lumen such as the large intestine, the capsule endoscope moves near an intraluminal wall surface and images captured by the imaging function cover in many cases only a narrow portion of areas near the intraluminal wall surface and thus desired images cannot be reliably obtained.

### SUMMARY OF THE INVENTION

An object of the present invention is to provide a capsule medical apparatus capable of capturing stable and desired wide-angle images along the flow of a fluid injected into a subject and a body-cavity observation method.

A capsule medical apparatus according to the present invention comprises a capsule casing having an approximately cylindrical trunk; a center of gravity being approximately in a volume center; and a specific gravity being approximately equal to that of a fluid injected into a body cavity.

The capsule medical apparatus according to the present invention further comprises an imaging unit disposed at least at one end of the capsule casing so that a longitudinal axis direction of the capsule casing and an optical axis direction approximately coincide with each other; and an illuminator for illuminating an imaging field.

In the capsule medical apparatus according to the present invention, the imaging unit and the illuminator are provided at both ends of the capsule casing.

The capsule medical apparatus according to the present invention comprises a power source or a battery for supplying power to the capsule medical apparatus, wherein a weight balance of the capsule medical apparatus is maintained by a disposition location thereof.

In the capsule medical apparatus according to the present invention, at least one end of the capsule casing in the longitudinal axis direction has an approximately hemispherical shape.

In the capsule medical apparatus according to the present invention, at least one end of the capsule casing where the imaging unit is disposed is transparent.

In the capsule medical apparatus according to the present invention, the body cavity is large intestine.

The capsule medical apparatus according to the present invention comprises a fluid resistor having resistance to the fluid flowing inside the body cavity.

In the capsule medical apparatus according to the present invention, the fluid resistor is a fin, a protrusion, a cutout, a hole, a groove, or a combination thereof.

In the capsule medical apparatus according to the present invention, the fluid resistor is a straightener for straightening a flow of the fluid.

In the capsule medical apparatus according to the present invention, the fluid resistor is a rotator causing rotational movement about the longitudinal axis.

In the capsule medical apparatus according to the present invention, the fluid resistor is an eccentric rotator causing eccentric rotational movement about the longitudinal axis.

In the capsule medical apparatus according to the present invention, the eccentric rotator has a different amount of rotation by one fluid resistor provided at a first end of the capsule casing from that by the other fluid resistor provided at a second end of the capsule casing.

The capsule medical apparatus according to the present invention comprises a vibrator which vibrates the capsule medical apparatus.

In the capsule medical apparatus according to the present invention, the vibrator is a motor or a magnet.

The capsule medical apparatus according to the present invention comprises a contact recognizer which senses that the capsule medical apparatus is in contact with an external object.

A body-cavity observation method according to the present invention comprises the steps of taking in a capsule medical apparatus; taking in a fluid whose specific gravity is approximately equal to that of the capsule medical apparatus; generating a flow rate of the fluid; and capturing images inside the body cavity while the capsule medical apparatus is drifting in the fluid.

In the body-cavity observation method according to the present invention, the step of generating the flow rate includes at least one of manual pressure, purgative intake, and postural change.

In the body-cavity observation method according to the present invention, the step of capturing images includes the step of causing the capsule medical apparatus to drift in a body cavity cross section where the flow rate is approximately maximum.

In the body-cavity observation method according to the present invention, the step of capturing images includes the step of straightening an orientation of image capturing by the capsule medical apparatus to the flow of the fluid.

In the body-cavity observation method according to the present invention, the step of capturing images includes the steps of detecting that the capsule medical apparatus is in contact with a body cavity wall surface; and capturing images in accordance with a result of the detection.

In the body-cavity observation method according to the present invention, the step of capturing images includes the steps of detecting that the capsule medical apparatus is in contact with a body cavity wall surface; causing the capsule medical apparatus to vibrate in accordance with a result of the detection; and capturing images while the vibration is not occurring.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1A is a diagram showing an outline configuration of a capsule endoscope in an embodiment of the present invention;

FIG. 1B is a diagram showing a first modification of the capsule endoscope shown in FIG. 1A obtained by disposing an imaging optical system of the capsule endoscope at both ends of the capsule casing;

FIG. 2 is a diagram schematically showing movement of the capsule endoscope shown in FIG. 1 inside a lumen;

FIG. 3 is a diagram showing an example of desired body-cavity images captured by the capsule endoscope shown in FIG. 1;

FIG. 4 is a diagram showing a second modification obtained by providing a straightener with through holes in the capsule endoscope;

FIG. 5 is a diagram showing a third modification obtained by providing a straightener with fins in the capsule endoscope;

FIG. 6 is a diagram showing a fourth modification obtained by providing a straightener with grooves in the capsule endoscope;

FIG. 7 is a diagram showing a fifth modification obtained by providing a rotator with through holes in the capsule endoscope;

FIG. 8 is a diagram showing a sixth modification obtained by providing a rotator with fins in the capsule endoscope;

FIG. 9 is a diagram showing a seventh modification obtained by providing a rotator with grooves in the capsule endoscope;

FIG. 10 is a diagram showing a eighth modification obtained by providing a rotator with finned cutouts in the capsule endoscope;

FIG. 11 is a diagram showing a ninth modification obtained by providing an eccentric rotator causing eccentric movement of the capsule endoscope;

FIG. 12A is a diagram showing a modification of a sectional shape of a through hole, a fin, a groove, or a cutout;

FIG. 12B is a diagram showing a modification of the sectional shape of a through hole, a fin, a groove, or a cutout;

FIG. 12C is a diagram showing a modification of the sectional shape of a through hole, a fin, a groove, or a cutout;

FIG. 12D is a diagram showing a modification of the sectional shape of a through hole, a fin, a groove, or a cutout;

FIG. 12E is a diagram showing a modification of the sectional shape of a through hole, a fin, a groove, or a cutout;

FIG. 12F is a diagram showing a modification of the sectional shape of a through hole, a fin, a groove, or a cutout;

FIG. 12G is a diagram showing a modification of the sectional shape of a through hole, a fin, a groove, or a cutout;

FIG. 12H is a diagram showing a modification of the sectional shape of a through hole, a fin, a groove, or a cutout;

FIG. 12I is a diagram showing a modification of the sectional shape of a through hole, a fin, a groove, or a cutout; and

FIG. 13 is a diagram showing a tenth modification obtained by providing a vibrator in the capsule endoscope.

### DETAILED DESCRIPTION OF THE PREFERRED EMBODIMENTS

An embodiment of a capsule medical apparatus according to the present invention will be described in detail below with reference to drawings. In the present embodiment, a capsule endoscope having at least an imaging function will be described as an exemplary capsule medical apparatus. However, the present invention is not limited to the present embodiment and can be carried out in various modifications without departing from the scope of the present invention.

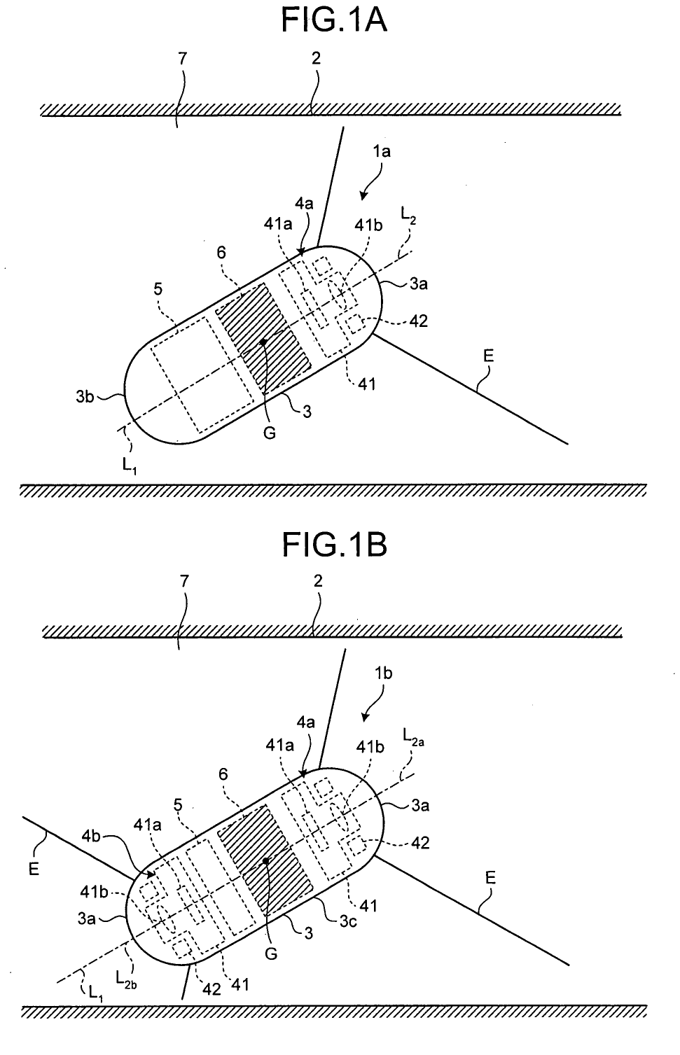

FIG. 1A is a diagram showing an outline configuration of a capsule endoscope, which is an embodiment of the capsule medical apparatus according to the present invention. A capsule endoscope 1 comprises a capsule casing 3 insertable into a body cavity of a subject 2, an imaging optical system 4a that is disposed inside the capsule casing 3 and can capture images in a front end direction, circuit systems 5 such as a control board, circuit components, and a transmitting antenna disposed inside the capsule casing 3, and a battery 6.

The capsule casing 3 is of such a size that it can be swallowed into a body through an oral cavity of the subject 2, and forms an exterior case that seals an inner part fluid-tightly by elastically fitting an approximately hemispherical end cover 3a having transparency or translucency and a closed-end cylindrical trunk cover 3b made of colored material to which visible light is not transparent.

Here, the capsule endoscope 1 of the present invention is used to obtain, for example, an inner wall of the large intestine as in-vivo images, and the capsule casing 3 has a specific gravity including incorporated components thereof roughly equal to that of a fluid 7 and a center of gravity G is located approximately in a volume center. As a result, the capsule endoscope 1 in the fluid 7 is unstable in its posture and position. The fluid 7 is a fluid that can be swallowed through the oral cavity of the subject 2 and is transparent to wavelengths of the imaging optical system 4a of the capsule endoscope 1 and, in the present embodiment, potable water or clyster whose specific gravity is roughly equal to 1 is exemplarily used. However, the specific gravity may be greater than 1 in the present embodiment.

The battery 6 is a heavy load among components of the capsule endoscope 1 and is disposed in approximately a central part of the capsule casing 3, and because it is heavy load, a weight balance can be maintained mainly by changing its position.

The imaging optical system 4a is comprised of an imaging unit 41 and an illuminator 42. The imaging unit 41 comprises an imaging device 41a such as a CCD or C-MOS imager that captures an image of an object by receiving a reflected light of the object by an illumination light of the illuminator 42 as an intra-subject image and an image formation lens 41b for forming an image of the object on the imaging device 41a on an axial center of the capsule casing 3 to obtain an image of an object as an intra-subject image.

The illuminator 42 is used for illuminating a imaging field E of the imaging unit 41 and is realized by a plurality of light sources, for example, LEDs radiating an irradiation light for illuminating an imaging region of an object via the end cover 3a. The plurality of LEDs are disposed around the imaging unit 41 with respect to an optical axis center of the imaging unit 41 so that the entire imaging field E is covered.

Meanwhile, a longitudinal axis center line L1 of the capsule endoscope 1 and an optical axis L2 of the imaging optical system coincide with each other in the present embodiment.

As already described above, the capsule endoscope 1 in the fluid 7 is unstable in its posture and position. As a result, the capsule endoscope 1 becomes, on the other hand, more susceptible to an influence of the flow of the fluid 7 and easier to move.

FIG. 1B is a diagram showing the configuration in which the imaging optical system 4a is disposed at both ends of the capsule casing 3 in the capsule endoscope, which is the embodiment of the capsule medical apparatus according to the present invention. In the present embodiment, the longitudinal axis center line L1 of a capsule endoscope 1b, an optical axis L2a of the imaging optical system 4a, and an optical axis L2b of an imaging optical system 4b coincide with each other.

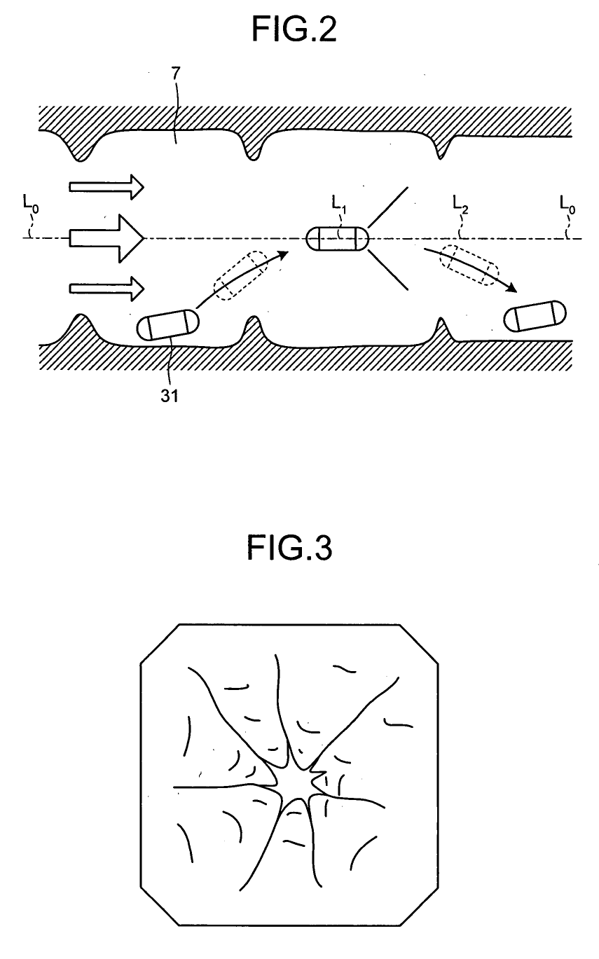

In a lumen of the large intestine for example, as shown in FIG. 2, the flow of the fluid 7 is fast near a luminal central axis L0 and slow near a luminal wall surface. Thus, a capsule endoscope 31 positioned near the wall surface is less susceptible to the flow of the fluid 7, but since the capsule endoscope 31 has a specific gravity roughly equal to that of the fluid 7 and its center of gravity G is located approximately in the volume center, the capsule endoscope 31 is unstable and susceptible also to a slow flow of the fluid 7 and thus can move easily. The flow of the fluid 7 may be a flow caused passively for example by gravity, peristaltic movement, and segmentation, or a flow caused actively for example by manual pressure, purgative intake, and postural change.

If the capsule endoscope 31 moves away from the luminal wall surface under an influence of the flow of the fluid 7, the capsule endoscope 31 approaches the luminal central axis L0 while moving along the flow due to a difference between a flow rate of a vicinity of the luminal central axis L0 and that of the luminal wall surface. After approaching the luminal central axis L0, the capsule endoscope 31 moves in a posture in which the longitudinal axis center line L1 is in line with the luminal central axis L0 because the capsule endoscope 31 has an approximately cylindrical shape stretching in the longitudinal axis direction and both ends have the approximately hemispherical shape. The capsule endoscope 31 is, so to speak, centered in the lumen and the optical axis L2 also moves in the direction of the luminal central axis L0. The capsule endoscope 31 near the luminal central axis L0 where the flow rate of the fluid 7 is fast is stable in its posture and position.

As a result, the capsule endoscope 13 can capture desired intraluminal images that can command a panoramic view of the luminal wall surface along the direction of the luminal central axis L0, that is, from a downstream direction or an upstream direction of the fluid 7. As shown in FIG. 3, the luminal central axis L0 is positioned in the center of a captured intraluminal image.

If a difference between a flow rate of the luminal central axis L0 and a vicinity of the luminal wall surface disappears, as shown in FIG. 2, the capsule endoscope 31 moves away from the vicinity of the luminal central axis L0 and becomes unstable.

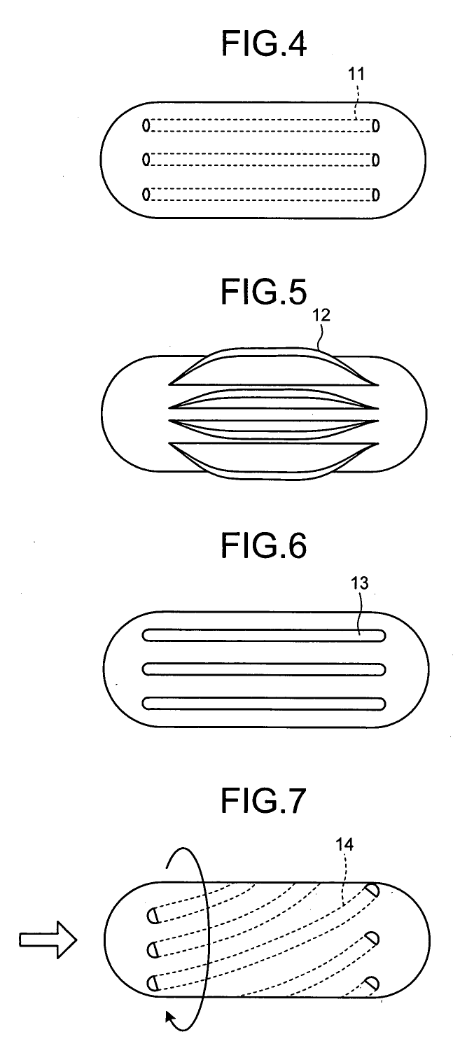

The capsule endoscopes 1a, 1b, and 31 described above are stable near the luminal central axis L0 where the flow rate of the fluid 7 is fast because the capsule casing itself has an elongated shape, but in order to further increase stability, a plurality of through holes 11 along the longitudinal axis direction of the capsule casing 3 shown in FIG. 4 may be provided to straighten the fluid 7. Through straightening of the fluid 7 by the through holes 11, the direction of flow of the fluid 7 and the longitudinal axis direction of the capsule endoscope 1 coincide with each other, resulting in a stable agreement of the optical axis L2 and the luminal central axis L0.

Similarly, as shown in FIG. 5, a plurality of fins 12 along the longitudinal axis direction of the capsule endoscope 1 may be provided on an outer surface of the capsule casing 3 to stabilize the posture of the capsule endoscope 1 through straightening of the fluid 7.

Also, as shown in FIG. 6, a plurality of grooves 13 along the longitudinal axis direction of the capsule endoscope 1 may be provided for straightening.

Straighteners such as the fins 12 for straightening the flow of the fluid 7 are provided in the capsule endoscopes shown in FIG. 4 to FIG. 6, but the posture of the capsule endoscope 1 may also be stabilized by rotating the capsule endoscope about the longitudinal axis.

In a capsule endoscope shown in FIG. 7, a plurality of through holes 14 are provided near the surface of the capsule casing and the through holes 14 are formed to be spiral about the longitudinal axis. The capsule endoscope receives thereby fluid resistance by the fluid 7 passing through the through holes 14 so that the capsule endoscope rotates about the longitudinal axis. The capsule endoscope can attain through the rotational movement a stable physical relationship in which the longitudinal axis runs along the fluid.

Instead of the through holes 14 shown in FIG. 7, fins 15, grooves 16, or finned cutouts 17 may be provided. Each of the fins 15, grooves 16, or finned cutouts 17 can stabilize the posture of the capsule endoscope by rotating the capsule endoscope about the longitudinal axis.

Straighteners are provided or the capsule endoscope is rotated in the embodiment described above to stabilize the posture of the capsule endoscope, but the present invention is not limited to such modifications of the present embodiment and the longitudinal axis direction of the capsule endoscope may be forced to change bit by bit while roughly stabilizing the posture of the capsule endoscope.

For example, as shown in FIG. 11, a plurality of fins 18a and a plurality of fins 18b for receiving fluid resistance of the fluid 7 may be provided at different end edges in such a way that the fluid resistance received by the fins 18a is different from that received by the fins 18b to produce different turning efforts at both end edges, causing eccentric movement about the longitudinal axis center line L1. By making this eccentric movement, images can be captured inside the lumen at substantially still wider angles.

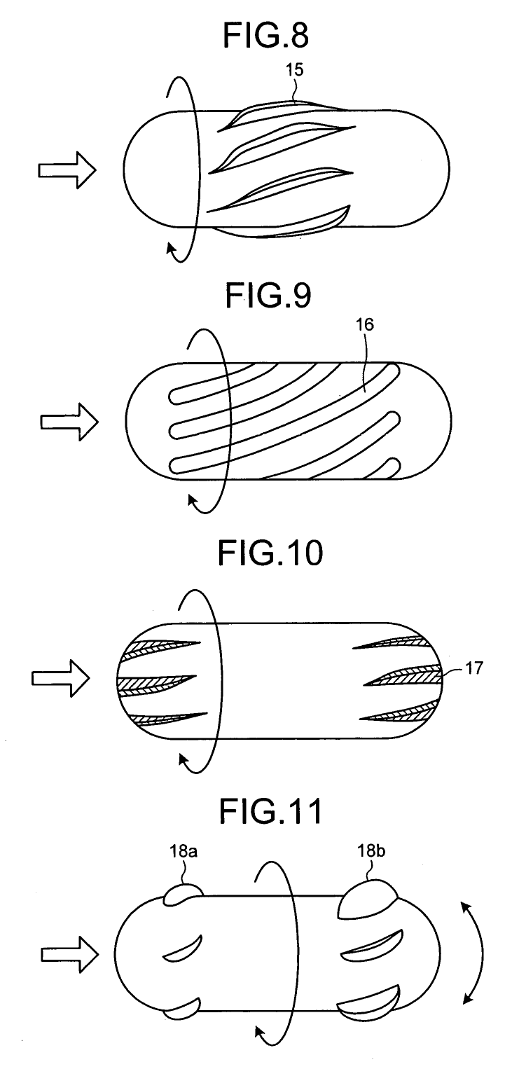

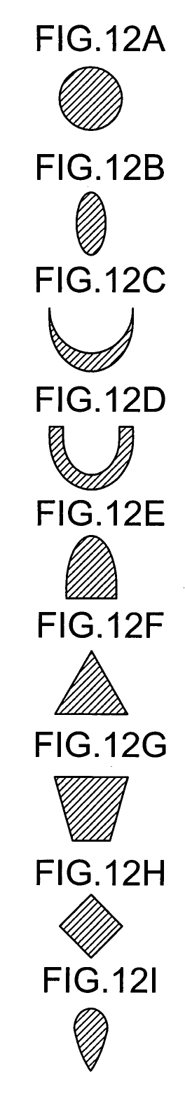

FIG. 12A to FIG. 12I show modifications of the sectional shape of the through holes, fins, grooves, and cutouts shown in FIG. 4 to FIG. 11. Each sectional shape can take any shape shown here or a combination of these shapes.

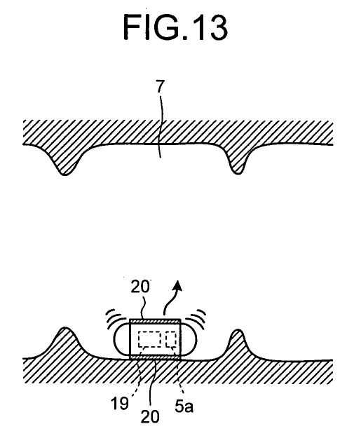

FIG. 13 shows a modification obtained by providing a vibrator 19 realized by an eccentric motor or the like, a control unit 5a for performing vibration control of the vibrator 19, and a contact recognizer 20 for sensing a contact state in which the capsule endoscope is in contact with a body cavity tissue in the above capsule endoscope. The whole capsule endoscope 1 is vibrated when no image is captured to make it easier to be separated from the wall surface of the lumen. Instead of the vibrator 19, a magnet may be provided. In this case, the control unit 5a is removed and the magnet may be caused to vibrate by applying an external oscillating magnetic field, thereby causing the capsule endoscope itself to vibrate. However, it is also preferable to perform a control operation such that the magnet is vibrated only when no image is captured. For example, the vibrator 19 is caused to vibrate when the contact recognizer 20 detects that the capsule endoscope is in contact with a body cavity wall. When the capsule endoscope separates from the body cavity wall due to the vibration and the contact recognizer 20 detects that the capsule endoscope is not in contact with a body cavity wall, the vibration of the vibrator 19 is stopped so that images can be captured. In this way, more stable images can be captured.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A capsule medical apparatus, comprising:
a capsule casing having an approximately cylindrical trunk;
a center of gravity being approximately in a volume center; and
a specific gravity being approximately equal to that of a fluid injected into a body cavity.

2. The capsule medical apparatus according to claim 1, further comprising:
an imaging unit disposed at least at one end of the capsule casing so that a longitudinal axis direction of the capsule casing and an optical axis direction approximately coincide with each other; and
an illuminator for illuminating an imaging field.

3. The capsule medical apparatus according to claim 2, wherein the imaging unit and the illuminator are provided at both ends of the capsule casing.

4. The capsule medical apparatus according to claim 1, comprising a power source or a battery for supplying power to the capsule medical apparatus, wherein a weight balance of the capsule medical apparatus is maintained by a disposition-location thereof.

5. The capsule medical apparatus according to claim 1, wherein at least one end of the capsule casing in the longitudinal axis direction has an approximately hemispherical shape.

6. The capsule medical apparatus according to claim 2, wherein at least one end of the capsule casing where the imaging unit is disposed is transparent.

7. The capsule medical apparatus according to claim 1, wherein the body cavity is large intestine.

8. The capsule medical apparatus according to claim 1, comprising a fluid resistor having resistance to the fluid flowing inside the body cavity.

9. The capsule medical apparatus according to claim 8, wherein the fluid resistor is a fin, a protrusion, a cutout, a hole, a groove, or a combination thereof.

10. The capsule medical apparatus according to claim 8, wherein the fluid resistor is a straightener for straightening a flow of the fluid.

11. The capsule medical apparatus according to claim 8, wherein the fluid resistor is a rotator causing rotational movement about the longitudinal axis.

12. The capsule medical apparatus according to claim 8, wherein the fluid resistor is an eccentric rotator causing eccentric rotational movement about the longitudinal axis.

13. The capsule medical apparatus according to claim 12, wherein the eccentric rotator has a different amount of rotation by one fluid resistor provided at a first end of the capsule casing from that by the other fluid resistor provided at a second end of the capsule casing.

14. The capsule medical apparatus according to claim 1, comprising a vibrator which vibrates the capsule medical apparatus.

15. The capsule medical apparatus according to claim 14, wherein the vibrator is a motor or a magnet.

16. The capsule medical apparatus according to claim 1, comprising a contact recognizer which senses that the capsule medical apparatus is in contact with an external object.

17. A body-cavity observation method comprising the steps of:
taking in a capsule medical apparatus;
taking in a fluid whose specific gravity is approximately equal to that of the capsule medical apparatus;
generating a flow rate of the fluid; and
capturing images inside the body cavity while the capsule medical apparatus is drifting in the fluid.

18. The body-cavity observation method according to claim 17, wherein the step of generating the flow rate includes at least one of manual pressure, purgative intake, and postural change.

19. The body-cavity observation method according to claim 17, wherein
the step of capturing images includes the step of causing the capsule medical apparatus to drift in a body cavity cross section where the flow rate is approximately maximum.

20. The body-cavity observation method according to claim 17, wherein
the step of capturing images includes the step of straightening an orientation of image capturing by the capsule medical apparatus to the flow of the fluid.

21. The body-cavity observation method according to claim 17, wherein
the step of capturing images includes the steps of
detecting that the capsule medical apparatus is in contact with a body cavity wall surface; and
capturing images in accordance with a result of the detection.

22. The body-cavity observation method according to claim 17, wherein
the step of capturing images includes the steps of
detecting that the capsule medical apparatus is in contact with a body cavity wall surface;
causing the capsule medical apparatus to vibrate in accordance with a result of the detection; and
capturing images while the vibration is not occurring.
